(19) United States
(12) Patent Application Publication
(10) Pub. No.: US 2015/0148113 A1
(43) Pub. Date: May 28, 2015

(54) PATIENT-SPECIFIC REHABILITATIVE VIDEO GAMES

(71) Applicant: BIOGAMING LTD., Petach Tikva (IL)

(72) Inventors: David Moshe KLEIN, Petach Tikva (IL); Eytan Sabari Majar, Karmei Yosef (IL)

(73) Assignee: BIOGAMING LTD

(21) Appl. No.: 14/611,309

(22) Filed: Feb. 2, 2015

Related U.S. Application Data
(63) Continuation-in-part of application No. 14/418,945, filed on Feb. 1, 2015.

(30) Foreign Application Priority Data
Mar. 6, 2013 (GB) 1304042.3

(51) Int. Cl. G06F 19/00; A61B 5/00; A63B 24/00; A63B 71/06; G06Q 50/22

(52) U.S. Cl. CPC G06F 19/3481; A63B 71/06; G06Q 50/22; A63B 24/0003; A61B 5/486
USPC 463/7

(57) ABSTRACT

A method for user specific, rehabilitative video gaming, receiving the following information: a rehabilitative therapy plan for the user, the rehabilitative therapy plan comprising multiple different physical exercises; a list of past games played by the user and exercises included in the games to determine a level of familiarity of the user with the games and exercises; past performance scores for each exercise performed by the user, then automatically translating the received information into a video game configured for execution by a motion recognition gaming system, by selecting a set of one or more games that include all the exercises required by the therapy plan; selecting a mode of operation for execution of the games by the user; selecting a help level for each exercise in the game; and enabling the user to conduct an interactive gaming session using the selected games.

### RELATED APPLICATIONS

The current application claims priority as a continuation in part under 5 U.S.C. 120 from U.S. patent application Ser. No. 14/418,945 filed on Feb. 1, 2015 the disclosure of which is incorporated herein by reference.

### FIELD OF THE INVENTION

The invention relates to patient-specific, rehabilitative video games.

### BACKGROUND

Decline in physical function is often associated with age-related impairments to overall health, or may be the result of injury or disease. Such a decline contributes to parallel declines in self-confidence, social interactions and community involvement. People with motor disabilities often experience limitations in fine motor control, strength, and range of motion. These deficits can dramatically limit their ability to perform daily tasks, such as dressing, hair combing, and bathing, independently. In addition, these deficits, as well as pain, can reduce participation in community and leisure activities, and even negatively impact occupation.

Participating in and complying with physical therapy, which usually includes repetitive exercises, is an essential part of the rehabilitation process which is aimed to help people with motor disabilities overcome the limitations they experience. However, it has been argued that most of the people with motor disabilities do not perform the exercises as recommended. People often cite a lack of motivation as an impediment to them performing the exercises regularly. Furthermore, the number of exercises in a therapy session is oftentimes insufficient. During rehabilitation, the therapist usually personally provides physical assistance and monitors whether each student's movements are reaching a specific standard. Thus, the therapist can only rehabilitate one patient at a time, or a small group of patients at most. Patients often lack enthusiasm to participate in the tedious rehabilitation process, resulting in continued muscle atrophy and insufficient muscle endurance.

Also, it is well known that adults and especially children get bored repeating the same movements. This can be problematic when an adult or a child has to exercise certain muscles during a post-trauma rehabilitation period. For example, special exercises are typically required after a person breaks his or her arm. It is hard to make this repetitive work interesting. Existing methods to help people during rehabilitation include games to encourage people, and especially children, to exercise more.

Therefore, it is highly advantageous for patients to perform rehabilitative physical therapy at home, using techniques to make repetitive physical exercises more entertaining. Uses of video games technologies are beginning to be explored as a commercially available means for delivering training and rehabilitation programs to patients in their own homes.

U.S. Pat. No. 6,712,692 to Basson et al. discloses a method for gathering information about movements of a person, which could be an adult or child. This information is mapped to one or more game controller commands. The game controller commands are coupled to a video game, and the videogame responds to the game controller commands as it would normally.

U.S. Pat. No. 7,996,793 to Latta et al. discloses Systems, methods and computer readable media for gesture recognizer system architecture. A recognizer engine is provided, which receives user motion data and provides that data to a plurality of filters. A filter corresponds to a gesture, which may then be tuned by application receiving information from the gesture recognizer so that the specific parameters of the gesture-such as arm acceleration for a throwing gesture may be set on a per-application level, or multiple times within a single application. Each filter may output to an application using it a confidence level that the corresponding gesture occurred, as well as further details about the user motion data.

U.S. Patent Application No. 2012/0190505A1 to Shavit et al. discloses a system for monitoring performance of a physical exercise routine comprises a Pilates exercise device enabling a user to perform the physical exercise routine, a plurality of motion and position sensors for generating sensory information that includes at least position and movements of a user performing the physical exercise routine; a database containing routine information representing at least an optimal execution of the physical exercise routine; a training module configured to separate from sensory information at least appearance of the Pilates exercise device, compare the separated sensory information to the routine information to detect at least dissimilarities between the sensory information and the routine information, wherein the dissimilarities indicate an incorrect execution of the physical exercise routine, the training module is further configured to feedback the user with instructions related to correcting the execution of the physical exercise routine by the user; and a display for displaying the feedback.

Smith et al. (2012) disclose an overview of the main videogame console systems (Nintendo Wii™, Sony Playstation® and Microsoft Xbox®) and discussion of some scenarios where they have been used for rehabilitation, assessment and training of functional ability in older adults. In particular, two issues that significantly impact functional independence in older adults are injury and disability resulting from stroke and falls. See S. T. Smith, D. Schoene, The use of Exercise-based Videogames for Training and Rehabilitation of Physical Function in Older Adults, Aging Health. 2012; 8(3):243-252.

Ganesan et al. (2012) disclose a project that aims to find the factors that play an important role in motivating older adults to maintain a physical exercise routine, a habit recommended by doctors but difficult to sustain. The initial data gathering includes an interview with an expert in aging and physical therapy, and a focus group with older adults on the topics of exercise and technology. Based on these data, an early prototype game has been implemented for the Microsoft Kinect that aims to help encourage older adults to exercise. The Kinect application has been tested for basic usability and found to be promising. Next steps include play-tests with older adults, iterative development of the game to add motivational features, and evaluation of the game's success in encouraging older adults to maintain an exercise regimen. See S. Ganesan, L. Anthony, Using the Kinect to encourage older adults to exercise: a prototype, in Extended Abstracts of the ACM Conference on Human Factors in Computing Systems (CHI'2012), Austin, Tex., 5 May 2012, p.2297-2302.

Lange et al. (2011) disclose that the use of the commercial video games as rehabilitation tools, such as the Nintendo WiiFit, has recently gained much interest in the physical therapy arena. Motion tracking controllers such as the Nintendo Wiimote are not sensitive enough to accurately measure performance in all components of balance. Additionally, users can figure out how to “cheat” inaccurate trackers by performing minimal movement (e.g. wrist twisting a Wiimote instead of a full arm swing). Physical rehabilitation requires accurate and appropriate tracking and feedback of performance. To this end, applications that leverage recent advances in commercial video game technology to provide full-body control of animated virtual characters are developed. A key component of the approach is the use of newly available low cost depth sensing camera technology that provides markerless full-body tracking on a conventional PC. The aim of the research was to develop and assess an interactive game-based rehabilitation tool for balance training of adults with neurological injury. See B. Lange, C. Y. Chang, E. Suma, B. Newman, A. S. Rizzo, M. Bolas, Development and evaluation of low cost game-based balance rehabilitation tool using the Microsoft Kinect sensor, 33rd Annual International Conference of the IEEE EMBS, 2011.

The foregoing examples of the related art and limitations related therewith are intended to be illustrative and not exclusive. Other limitations of the related art will become apparent to those of skill in the art upon a reading of the specification and a study of the figures.

### SUMMARY

An aspect of an embodiment of the disclosure relates to a system and method for preparing a user specific rehabilitative video game. The system receives a therapy plan from a therapist wherein the plan requires that the user perform at least a specific set of exercises. Additionally the system receives a list of past games played by the user and exercises included in the games performed by the user to determine a level of familiarity of the user with the games and exercises. The system further receives past performance scores for each exercise performed by the user.

The system uses the received information to automatically translate the information into a set of video games for executing by a motion recognition gaming system. The translating includes:

Selecting a set of one or more games that include all the exercises required by the therapy plan;

Selecting a mode of operation for execution of the games by the user, based on the level of familiarity of the user with the games;

Selecting a help level for each exercise in the game, based on the past performance scores.

In some embodiments of the disclosure, the therapist communicates with the gaming system via a web site at a web server. Optionally, the translation process may be performed at the web server or at the user's gaming system.

There is thus provided according to an exemplary embodiment of the disclosure, a method for user specific, rehabilitative video gaming, the method comprising:

receiving the following information:

a. a rehabilitative therapy plan for the user, the rehabilitative therapy plan comprising multiple different physical exercises;

b. a list of past games played by the user and exercises included in the games to determine a level of familiarity of the user with the games and exercises;

c. past performance scores for each exercise performed by the user;

automatically translating the received information into a set of video games configured for execution by a motion recognition gaming system, wherein said automatically translating comprises:

selecting a set of one or more games that include all the exercises required by the therapy plan;

selecting a mode of operation for execution of the games by the user, based on the level of familiarity of the user with the games;

selecting a help level for each exercise in the game, based on the past performance scores; and

enabling the user to conduct an interactive gaming session using the selected games, by operating the motion recognition gaming system to recognize motion of the patient.

In an exemplary embodiment of the disclosure, the set of one or more games include exercises that were not required by the therapy plan. Optionally, the translating takes into account user limitations to exclude certain types of exercises from the set of one or more games. In an exemplary embodiment of the disclosure, the modes of operation include a beginner mode, an experienced user mode and a veteran mode. Optionally, the mode defines a level of providing the user with guidance during use of the games. Alternatively or additionally, the mode defines a level of requiring compliance with the requirements of the game and a level of required accuracy in performing the exercises. In an exemplary embodiment of the disclosure, an exercise in which the user has a high score will have a low help level and an exercise in which the user has a low score will have a high help level. Optionally, the help level of an exercise determines if the help for the exercise will be provided as a written message on a screen, a verbal message, a visual pictorial message, a video or a combination of the above. In an exemplary embodiment of the disclosure, the translating determines a portion of repetitions for performing each exercise from the therapy plan for each game from the set of one or more games. Optionally, the translating determines an order for performing exercises for each game. In an exemplary embodiment of the disclosure, the one or more physical exercises are selected from the group consisting of: hip flexion, jump, single-leg jump, leg stance, squat, single-leg squat, lunge, side lunge, forward kick, leg pendulum, lateral weight transfer, forward punch, shoulder flexion, shoulder abduction, shoulder upward press, side arm circle, diagonal arm movement, side punch, shoulder rotation with hands on body sides, shoulder rotation starting with abduction, and elbow flexion. In an exemplary embodiment of the disclosure, the receiving and the automatically translating are performed by a web server accessible by (a) a therapist who prescribes the rehabilitative therapy plan and (b) the motion recognition gaming system.

There is further provided according to an exemplary embodiment of the disclosure a non-transitory computer readable medium for storing a computer program for executing with a processor and memory the above method.

There is further provided according to an exemplary embodiment of the disclosure, a computing system configured for user specific, rehabilitative video gaming, the system comprising:

a communication module configured to receive:

a. a rehabilitative therapy plan for the user, the rehabilitative therapy plan comprising multiple different physical exercises;

b. a list of past games played by the user and exercises included in the games to determine a level of familiarity of the user with the games and exercises;

c. past performance scores for each exercise performed by the user;

a non-transient memory comprising instructions for automatically translating the therapy plan to a video game configured for execution by a motion recognition gaming system, wherein the automatically translating comprises:

selecting a set of one or more games that include all the exercises required by the therapy plan;

selecting a mode of operation for execution of the games by the user, based on the level of familiarity of the user with the games;

selecting a help level for each exercise in the game, based on the past performance scores; and

a hardware processor configured to execute the instructions, so as to generate the video games.

In an exemplary embodiment of the disclosure, the communication module is further configured to transmit the video games to the motion recognition gaming system. Alternatively, the computing system further comprises a motion recognition gaming system to recognize motion of the patient and conduct an interactive gaming session with the user using the selected games.

In an exemplary embodiment of the disclosure, the translating determines a portion of repetitions for performing each exercise from the therapy plan for each game from the set of one or more games. Optionally, the translating determines an order for performing exercises for each game.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows a block diagram of a computing system, in accordance with some embodiments;

FIG. 2 shows an example of a level formula on a schematic timeline, in accordance with some embodiments;

FIG. 3 shows an example of an exercise formula for hip flexion for one side, on a schematic timeline, in accordance with some embodiments;

FIG. 4 shows an example of an exercise formula for hip flexion for two sides, on a schematic timeline, in accordance with some embodiments;

FIG. 5 shows an example of an exercise formula for single leg stance for one side, on a schematic timeline, in accordance with some embodiments;

FIG. 6 shows an example of an exercise formula for single leg stance for two sides, on a schematic timeline, in accordance with some embodiments;

FIG. 7 shows an example of an exercise formula for single leg jump for one side, on a schematic timeline, in accordance with some embodiments;

FIG. 8 shows an example of an exercise formula for a single leg jump for two sides, on a schematic timeline, in accordance with some embodiments;

FIG. 9 shows an example of an exercise formula for a double leg jump, on a schematic timeline, in accordance with some embodiments;

FIG. 10 shows an example of a video game level on a schematic timeline, in accordance with some embodiments;

FIG. 11 shows an example of a dedicated web page which summarizes information on a certain patient, in accordance with some embodiments;

FIG. 12 shows an example of a dedicated web page which is utilized by the therapist to construct a therapy plan for a certain patient, in accordance with some embodiments;

FIG. 13 shows an illustration of a structured light method for depth recognition, in accordance with some embodiments;

FIG. 14 shows a top view 2D illustration of a triangulation calculation used for determining a pixel depth, in accordance with some embodiments;

FIG. 15 shows an illustration of a human primary body parts and joints, in accordance with some embodiments;

FIG. 16 shows an example of one video game level screen shot, in accordance with some embodiments;

FIG. 17 shows an example of another video game level screen shot, in accordance with some embodiments;

FIG. 18 shows a schematic flow diagram of a method of creating a user specific therapeutic game, in accordance with some embodiments;

FIG. 19 shows a schematic flow diagram of a method of monitoring a user specific therapeutic game, in accordance with some embodiments; and

FIG. 20 shows a schematic diagram of feedback messages for a user when required to perform a specific exercise, in accordance with some embodiments.

### DETAILED DESCRIPTION

Disclosed herein is a method for the automatic generation of patient-specific levels of video games, based on a physiotherapeutic and/or rehabilitative prescription given to the patient (also “user”).

Conventionally, people who require rehabilitative therapy, such as accident victims who suffered physical damages and need physiotherapeutic treatment, elderly people who suffer from degenerative diseases, children who suffer from physically-limiting cerebral palsy, etc., arrive to a rehabilitation center, meet with a therapist who prescribes a therapy plan for them, and execute the plan at the rehabilitation center and/or at home. In many cases, the therapy plan comprises of repeatedly-performed physical exercises, with or without therapist supervision. The plan normally extends over multiple appointments, when in each appointment the therapist may monitor the patient's progress and raise the difficulty level of the exercises. This conventional method has a few drawbacks: it requires the patient's arrival to the rehabilitation center, at least for a portion of the plan, which may be time consuming and difficult for some people (e.g. elderly people, small children, etc,), it often involves repetitive and boring activity, which may lead to lack of motivation and abandonment of the plan, and may limit the therapist to treat a rather small number of patients.

Thus, a method which may allow executing a therapy plan in the form of a video game, at the convenience of the patient's home, with easy communication between therapists and patients for plan prescribing and progress monitoring, may be highly advantageous to both therapists and patients. Moreover, a method which combines the aforementioned advantages while providing for patient-specific video games, rather than generic video games, is also of great significance.

### GLOSSARY

Video game: a game for playing by a human player, where the main interface to the player is visual content displayed using a monitor, for example. A video game may be executed by a computing device such as a personal computer (PC) or a dedicated gaming console, which may be connected to an output display such as a television screen, and to an input controller such as a handheld controller, a motion recognition device, etc.

Level of video game: a confined part of a video game, with a defined beginning and end. Usually, a video game includes multiple levels, where each level may involve a higher difficulty level and require more effort from the player.

Video game controller: a hardware part of a user interface (UI) used by the player to interact with the PC or gaming console.

Kinetic sensor: a type of a video game controller which allows the user to interact with the PC or gaming console by way of recognizing the user's body motion. Examples include handheld sensors which are physically moved by the user, body-attachable sensors, cameras which detect the user's motion, etc.

Motion recognition device: a type of a kinetic sensor, being an electronic apparatus used for remote (i.e. without physical contact with the player's body) sensing of a player's motions, and translating them to signals that can be input to the game console and used by the video game to react to the player motion and form interactive gaming.

Motion recognition game system: a system including a computer (e.g. a PC or a game console), and a motion recognition device.

Video game interaction: the way the user instructs the video game what he or she wishes to do in the game. The interaction can be, for example, mouse interaction, controller interaction, touch interaction, close range camera interaction or long range camera interaction.

Gesture: a physical movement of one or more body parts of a player, which may be recognized by the motion recognition device.

Exercise: a physical activity of a specific type, done for a certain rehabilitative purpose. An exercise may be comprised of one or more gestures. For example, the exercise referred to as “lunge”, in which one leg is moved forward abruptly, may be used to strengthen the quadriceps muscle, and the exercise referred to as “leg stance” may be used to improve stability, etc.

Repetition (also “instance”): one performance of a certain exercise. For example, one repetition of a leg stance exercise includes gestures which begin with lifting one leg in the air, maintaining the leg in the air for a specified period of time, and placing the leg back on the ground.

Intermission: A period of time between two consecutive repetitions of an exercise, during which period the player may rest.

In accordance with present embodiments, a method for generating a patient-specific, rehabilitative video game level may include: receiving a physiotherapeutic prescription (also “treatment plan”) from a therapist for a specific patient, automatically translating the prescription to a video game level customized to the patient, and allowing interactive gaming by utilizing a motion recognition device to recognize patient motion.

The exercises are part of the video game interactions. In the video game, various game features induce the users to perform certain gestures, which are designed to constitute, de-facto, physiotherapeutic and/or rehabilitative exercises. For example, a video game may be based on the principle of displaying a character which is a “double” of the patient on screen, in what is often referred to as a “third-person” game, and making the double mimic the gestures performed by the patient and recognized by the motion recognition device. The double may then be faced with different game challenges requiring the double, and hence the patient, to perform different gestures to overcome the challenges. For instance, an obstacle may induce the user to lift a leg or to jump, etc. A video game may also induce the patient to exercise in a more permissive manner, such as by letting the user to pick what exercised to perform and/or when to perform them, and endowing scores to the patient based on certain performance parameters.

Optionally, the therapist inputs his or her prescription for a certain patient via a dedicated web site, which may also allow management of communication between therapists and patients. The prescription may include one or more different exercises such as hip flexion (single-leg weight bearing), jump, single-leg jump, leg stance, squat, single-leg squat, lunge, side lunge, forward kick, leg pendulum, lateral weight transfer, forward punch, shoulder flexion, shoulder abduction, shoulder upward press, side arm circle, diagonal arm movement, side punch, shoulder rotation with hands on the sides, shoulder rotation starting with abduction, elbow flexion, etc., as known in the field of physiotherapy and physical rehabilitation. Each exercise may be defined with a certain number of repetitions and/or with reference to a single side of the sagital plane (right or left) or for both sides.

The present method may then automatically translate the therapist prescription to a newly-generated level of a video game, customized to that certain patient for whom the prescription was intended. Optionally, the translation utilizes specific rules of action intensity, for supporting proper exercising and enhancing gaming experience. For example, within a certain game level, the action intensity may gradually increase. It may be done by increasing exercise repetition rate, reducing idle intermissions and/or duration between exercises, etc.

Different games may also be selected according to the nature of the exercises in the prescription. The patient, in turn, may play the game while the motion recognition device captures his or her actions in a way that may allow the patient to see his actions on the screen, receive positive feedbacks for exercise completion, etc. One example for a suitable motion recognition device is the Microsft Corp. Kinect, a device for the Xbox 360 video game console and Windows PCs. Based around a webcam-style add-on peripheral for the Xbox 360 console, the Kinect enables users to control and interact with the Xbox 360 using a kinetic UI, without the need to touch a game controller, through a natural user interface using physical gestures.

The present video game may also be adapted to other gaming consoles, such as Sony PlayStation, Nintendo Wii, etc., and the motion recognition device may be a standard device for these or other gaming consoles.

Unless specifically stated otherwise, as apparent from the following discussions, it is appreciated that throughout the specification discussions utilizing terms such as “processing”, “computing”, “calculating”, “determining”, or the like, refer to the action and/or process of a computing system or a similar electronic computing device, that manipulate and/or transform data represented as physical, such as electronic, quantities within the computing system's registers and/or memories into other data similarly represented as physical quantities within the computing system's memories, registers or other such.

Some embodiments may be implemented, for example, using a computer-readable medium or article which may store an instruction or a set of instructions that, if executed by a computer (for example, by a hardware processor and/or by other suitable machines), cause the computer to perform a method and/or operations in accordance with embodiments of the invention. Such a computer may include, for example, any suitable processing platform, computing platform, computing device, processing device, computing system, processing system, computer, processor, gaming console or the like, and may be implemented using any suitable combination of hardware and/or software. The computer-readable medium or article may include, for example, any type of disk including floppy disks, optical disks, CD-ROMs, magnetic-optical disks, read-only memories (ROMs), random access memories (RAMs), flash memories, electrically programmable read-only memories (EPROMs), electrically erasable and programmable read only memories (EEPROMs), magnetic or optical cards, or any other type of media suitable for storing electronic instructions, and capable of being coupled to a computer system bus.

The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, such as C, C++, C#, Java, BASIC, Pascal, Fortran, Cobol, assembly language, machine code, or the like.

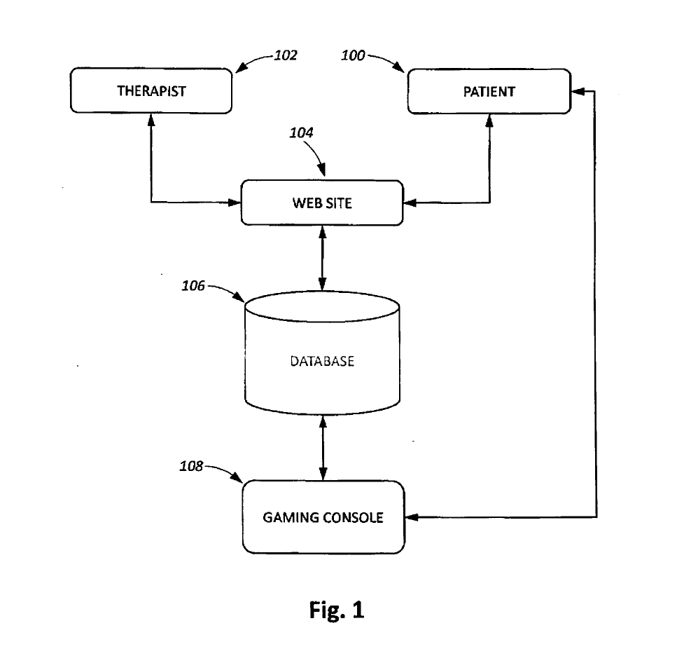

The present method may be better understood with reference to the accompanying figures. Reference is now made to FIG. 1, which shows a block diagram of a system operative according to the method. The therapist (102) may logon to the dedicated web site (104), communicate with patients (100), prescribe therapy plans (also referred to as “prescriptions” or “treatment plans”), and monitor patient progress. Web site (104) may receive the prescribed plan and store it in a dedicated database (106). The therapy plan may then be automatically translated to a video game level. When patient (100) activates his or her video game, the new level, or instructions for generating the new level, may be downloaded from web site (104) over a network to his or her computer (e.g. gaming console) (108) and he or she may play this new level. Since the game may be interactive, the motion recognition device may monitor the patient movements for storing patient results and progress, and or for providing real time feedback during the gameplay, such as in the form of score accumulation. The results, in turn, may be sent to database (106) for storage and may be available for viewing on web site (104) by therapist (102) for monitoring patient (100) progress, and to patient (100) for receiving feedback.

According to some embodiments, the automatic translation of the therapy plan to a video game level may apply the following rules: First, a chronologically-increasing action intensity within the level, to provide for an interesting, appealing and fun gaming exercise. Second, alternating sections within the same level, each section including one more repetitions of a certain exercise, also contributes to user interest and prevention of user burnout and boredom. The alternating sections may also contribute to proper action and rest cycles for the muscles, and to make the game more unexpected and by that enhance the patient gaming experience.

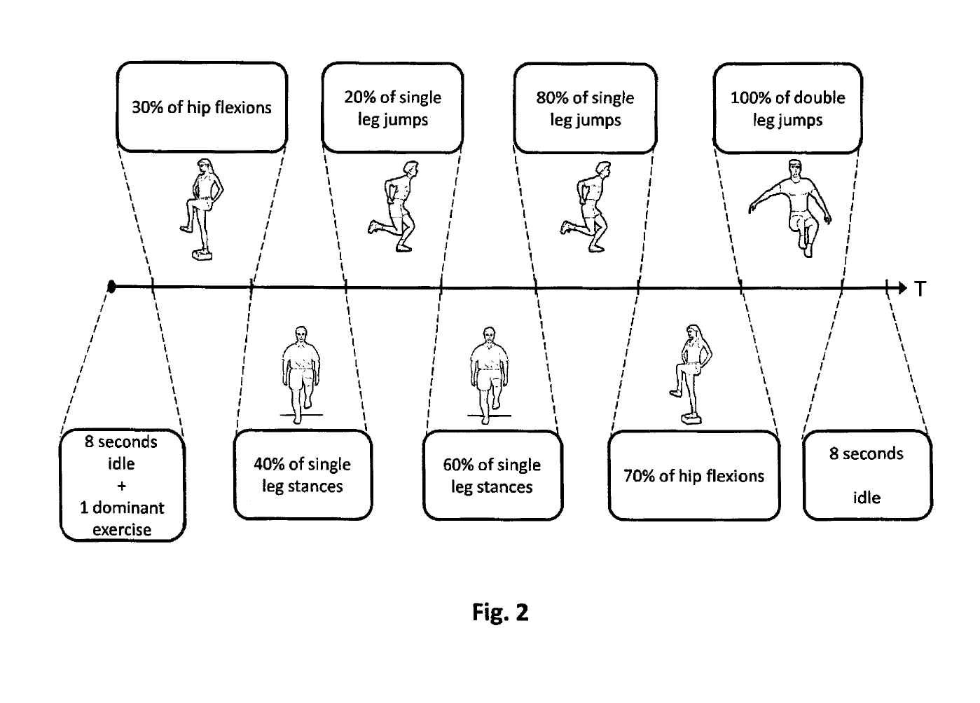

The division of the different exercises prescribed in the therapy plan to alternating sections, each including one or more repetitions of an exercise type, may be done by an advantageous level formula. Reference is now made to FIG. 2, which shows an example of a level formula on a schematic timeline. The level may start with a short idle period, such as a few seconds of idle, to let the patient prepare to gameplay. Optionally, one instance of the dominating exercise in that level may be performed in that idle period, implying to the patient what is going to be the nature of the level. Hip flexions may be divided to two sections: 30% at the level beginning, and 70% later on. Single leg stances may be divided to two sections: 20% at the beginning and 80% later on. Single leg jumps may be divided to two sections: 40% at the beginning and 60% afterwards. If possible, single leg jumps may be performed immediately after single leg stances, since single leg jumps are more convenient to accomplish when the opposing leg is already lifted. 100% of the double leg jumps may be performed at the last section of the level, before the idle section, since they are usually the hardest exercise of all. The level may end with a few seconds of idle.

Within a certain section of a certain exercise, the rule of increasing action intensity may be also applied. The division of the exercise instances within a section may be done by an exercise formula based on utilizing a scoring scheme. This scoring scheme need not be confused with regular game scoring which is shown to the patient as part of the game; rather, this scoring scheme is used during the level generation, in order to automatically construct the level out of the prescription. The exercise formula may divide each section into building blocks referred to as sub-sections, where each sub-section may contain multiple, for example five, instances of the exercise, and may be more intense than the previous sub-section, by way of increasing instance rate per time period (i.e. reducing intermissions), reducing idle times, etc.

An intensity curve of a section may be constructed based on the aforementioned scoring scheme, wherein a section is constructed by attaching together one or more predetermined sub-sections. Generally, a section may begin with the easiest predetermined sub-section. Then, this sub-section may be either repeated, or a next, harder sub-section may be added to the section, based on the scoring scheme.

In the course of the scoring scheme, each predetermined sub-section may be assigned a score which is accumulated each time after the sub-section is appended to the section. This score has another purpose, which is to indicate how many accumulated scores are required to skip to the next sub-section. This score becomes higher as the sub-sections become harder. When constructing a section, a next sub-section is appended only if the score assigned to it has already been accumulated by the previous appending of easier sub-sections. For example, the entrance score for the first sub-section may be 100, for the second sub-section 200, and so on and so forth.

When first starting to construct a section, a certain initial score may be provided, since no actual scores have been accumulated yet. The initial score may be determined according to the therapist's determination of the desired intensity of the section. For example, if the therapist wants the intensity level to be medium, the initial section score might be 0. If the therapist wants the intensity level to be low, the initial section score might be set to (−200), so the first, easiest sub-section is appended more than once before enough scores are accumulated to advance to the next sub-section. If the therapist wants the intensity level to be high, the initial section score might be set to 200, so the section starts from a later and harder sub-section.

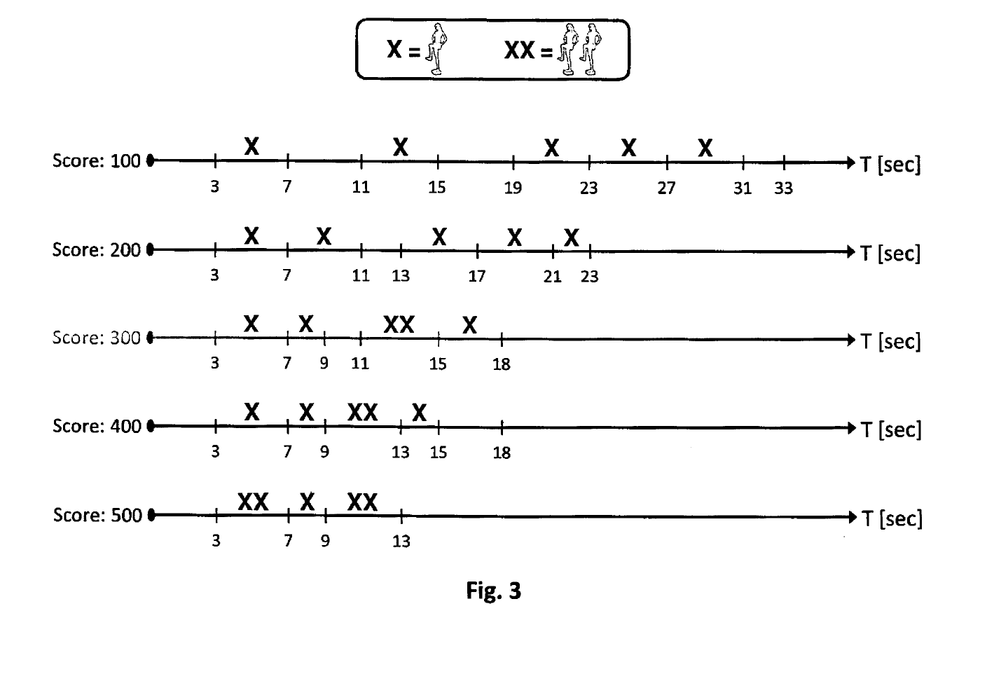

Reference is now made to FIG. 3, which shows an example of an exercise formula used to construct a section of single-side hip flexion. The example is shown using five schematic timelines, each representing a sub-section, wherein the sub-sections are arranged from the easiest (topmost) to the hardest (lowermost). Each sub-section has a score, and the hip flexion instances are depicted on the timeline by X marks. Sometimes, a double instance of the exercise may appear, depicted by an XX mark, which may require more effort from the patient. For example, in the first sub-section, the patient performs 3 seconds idle time, one hip flexion within the next 4 seconds, 4 seconds idle (i.e. “intermission”), one hip flexion within the next 4 seconds, etc. Upon finishing this sub-section, a score of 100 is accumulated. If the initial score was 0, it means that a score of 100 has been achieved, and the next sub-section may now be appended. After finishing the first sub-section (and first 5 hip flexions), if the patient has more than 5 hip flexions prescribed in his therapy plan, he may then perform the second sub-section. It should be noted, however, that a number different from 5 repetitions may be included in each sub-section. Whatever the number is, the total number of repetitions prescribed by the therapist may be divided among sub-sections. If, for example, after appending one or more sub-sections, the remaining amount of prescribed repetitions is less than 5 (or a different, preset number), the next sub-section may be performed in full, thereby slightly exceeding the prescribed number of repetitions.

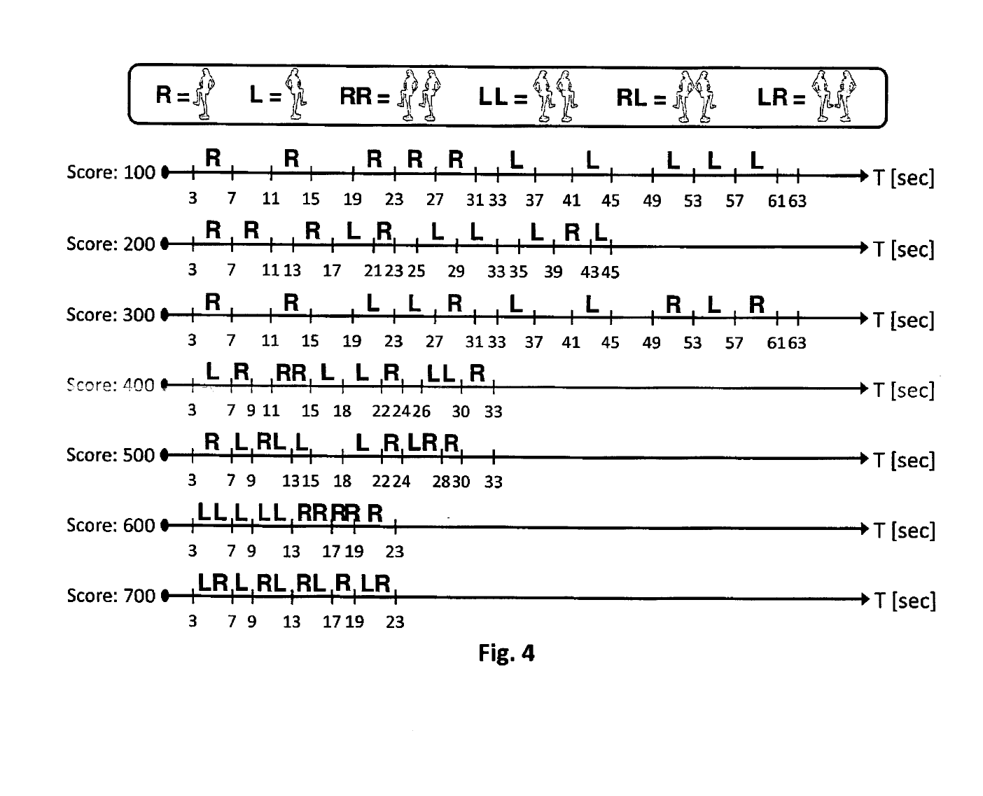

Reference is now made to FIG. 4, which shows an example of an exercise formula for two-sides hip flexion on a schematic timeline. Each sub-section has a score, and the hip flexion instances are depicted on the timeline by L (=Left) and R (=Right) marks. The left and right hip flexions may be performed alternately, to enhance the gaming experience. For example, in the first sub-section, the patient performs 3 seconds idle time, one right hip flexion within the next 4 seconds, 4 seconds idle, one right hip flexion within the next 4 seconds, etc. After finishing the first sub-section (and first 5 right and 5 left hip flexions), if the patient has more than 5 right and 5 left hip flexions prescribed in his therapy plan, he then may perform the second sub-section, etc. As one can see, the action intensity may increase upon advancing in sub-sections. Sometimes, a double instance of the exercise may appear, depicted by RR (Right+Right), LL (Left+Left), LR (Left+Right) or RL (Right+Left) mark, which may require more effort from the patient. Moreover, activity and idle sections may alternate within the sub-sections.

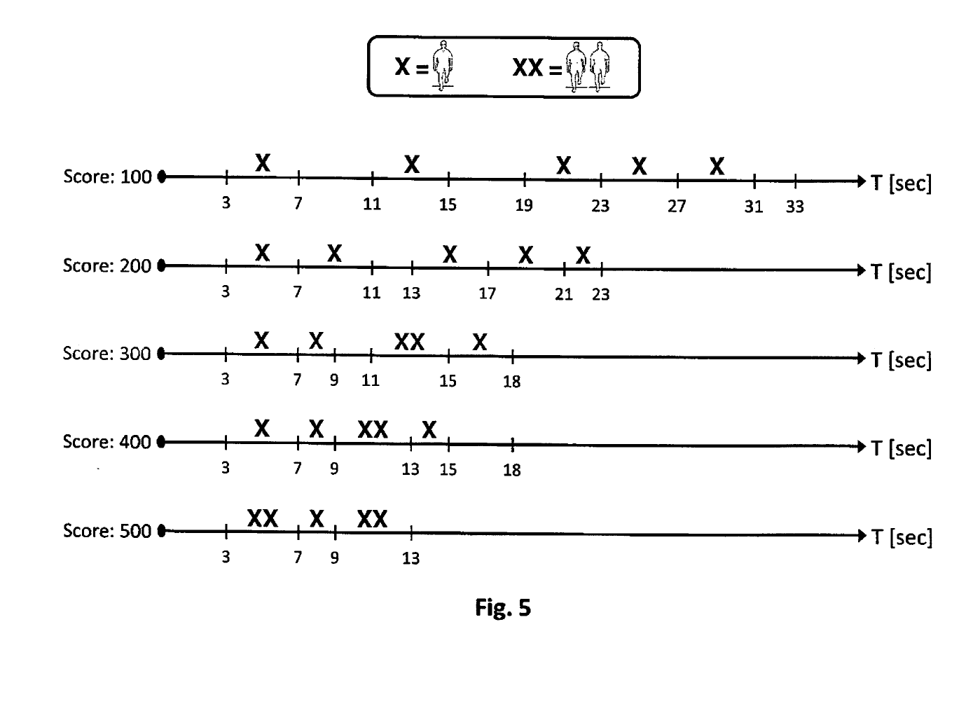

Reference is now made to FIG. 5, which shows an example of an exercise formula for a one-side leg stance on a schematic timeline. Each sub-section has a score, and the single leg stance instances are depicted on the timeline by X marks. For example, in the first sub-section, the patient performs 3 seconds idle time, one single leg stance within the next 4 seconds, 4 seconds idle, one single leg stance within the next 4 seconds, etc. After finishing the first sub-section (and first 5 single leg stances), if the patient has more than 5 single leg stances prescribed in his therapy plan, he then may perform the second sub-section, etc. As one can see, the action intensity may increase upon advancing in sub-sections. Sometimes, a double instance of the exercise may appear, depicted by XX mark, which may require more effort from the patient. Moreover, activity and idle sections may alternate within the sub-sections.

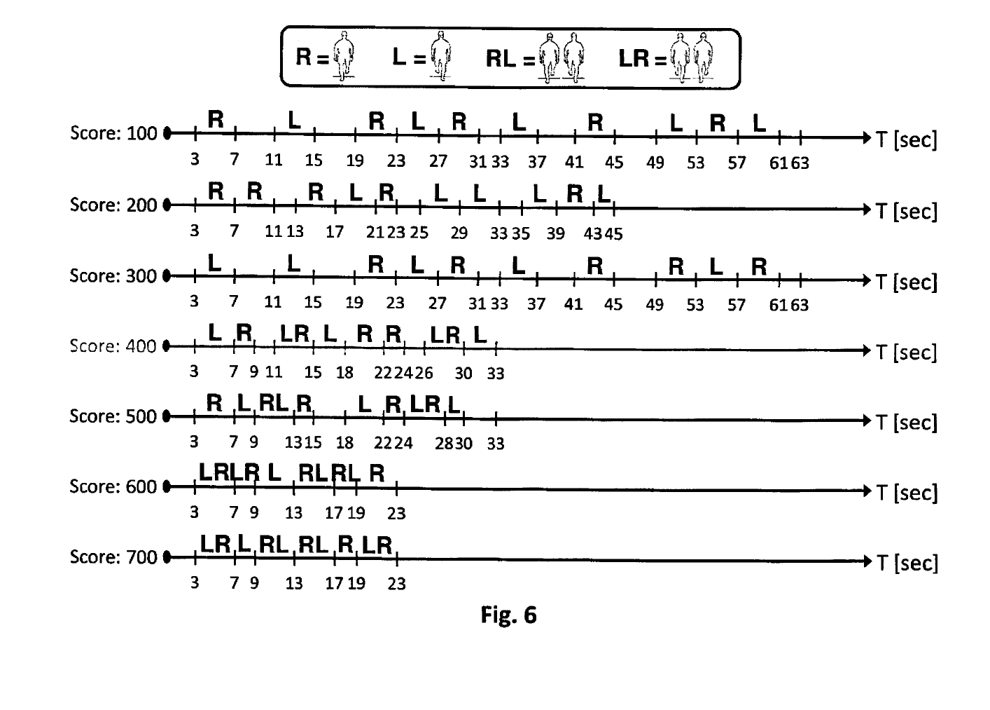

Reference is now made to FIG. 6, which shows an example of an exercise formula for single leg stance for two sides, on a schematic timeline. Each sub-section has a score, and the single leg stance instances are depicted on the timeline by L (=Left) and R (=Right) marks. The left and right single leg stances may be performed alternately, to enhance the gaming experience. For example, in the first sub-section, the patient performs 3 seconds idle time, one right single leg stance within the next 4 seconds, 4 seconds idle, one left single leg stance within the next 4 seconds, etc. After finishing the first sub-section (and first 5 right and 5 left single leg stances), if the patient has more than 5 right and 5 left single leg stances prescribed in his therapy plan, he then may perform the second sub-section, etc, As one can see, the action intensity may increase upon advancing in sub-sections. Sometimes a double instance of the exercise may appear, depicted by LR (Left+Right) or RL (Right+Left) mark, which may require more effort from the patient. Moreover, activity and idle sections may alternate within the sub-sections.

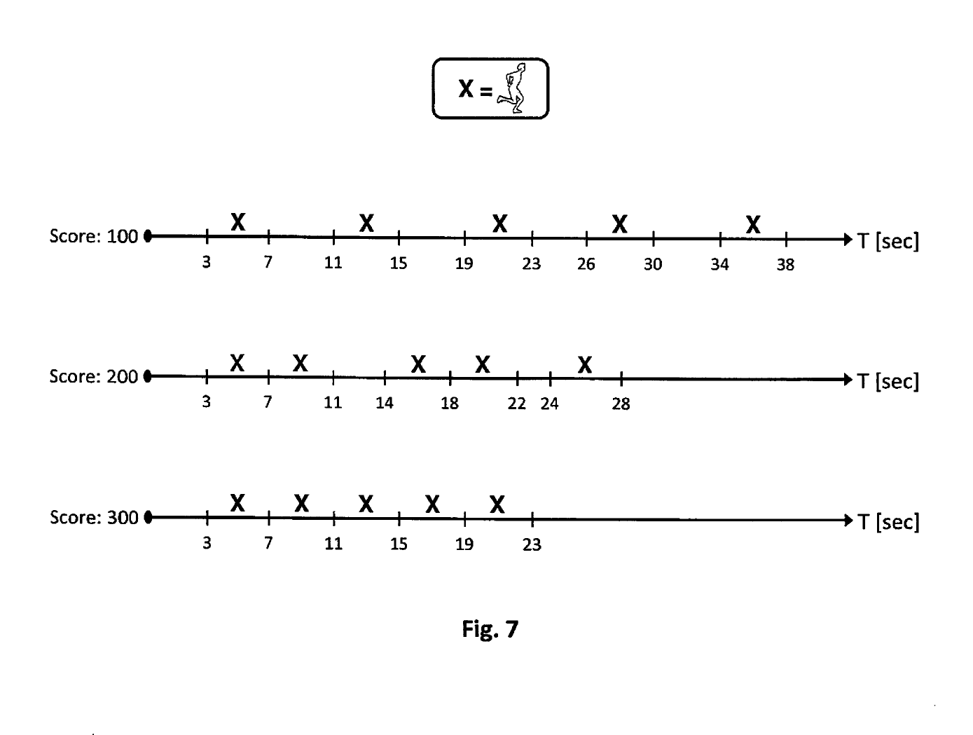

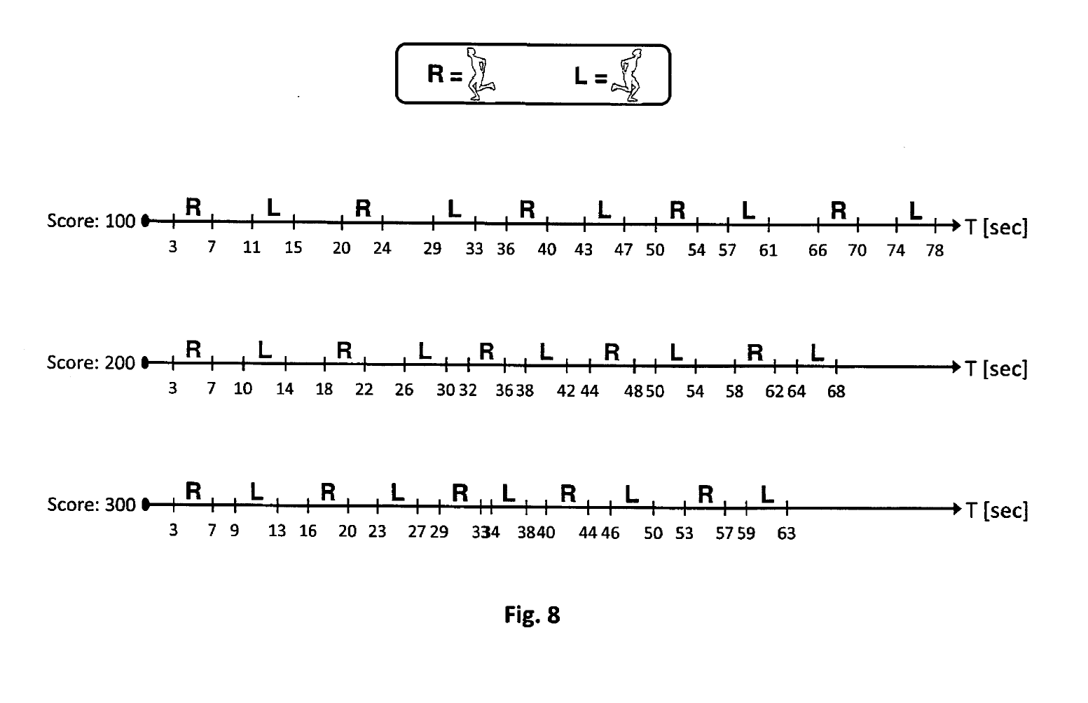

Reference is now made to FIG. 7, which shows an example of an exercise formula for a single leg, one-side jump, on a schematic timeline. Each sub-section has a score, and the single leg jump instances are depicted on the timeline by X marks. For example, in the first sub-section, the patient performs 3 seconds idle time, one single leg jump within the next 4 seconds, 4 seconds idle, one single leg jump within the next 4 seconds, etc. After finishing the first sub-section (and first 5 single leg jumps), if the patient has more than 5 single leg jumps prescribed in his therapy plan, he then may perform the second sub-section, etc, As one can see, the action intensity may increase upon advancing in sub-sections. Moreover, activity and idle sections may alternate within the sub-sections. Reference is now made to FIG. 8, which shows an example of exercise formula for single leg jump for two sides on a schematic timeline. Each sub-section has a score, and the single leg jump instances are depicted on the timeline by L (=Left) and R (=Right) marks. The left and right single leg jumps may be performed alternately, to enhance the gaming experience. For example, in the first sub-section, the patient performs 3 seconds idle time, one right single leg jump within the next 4 seconds, 4 seconds idle, one left single leg jump within the next 4 seconds, etc. After finishing the first sub-section (and first 5 right and 5 left single leg jumps), if the patient has more than 5 right and 5 left single leg jumps prescribed in his therapy plan, he then may perform the second sub-section, etc, As one can see, the action intensity may increase upon advancing in sub-sections. Moreover, activity and idle sections may alternate within the sub-sections.

Single leg jumps may be attached to single leg stances, i.e. some or every single leg stance may be immediately followed by a single leg jump. If the therapy plan includes single leg jumps that cannot be attached to single leg stances (e.g. there are more prescribed single leg jumps than prescribed single leg stances), the single leg jumps may be performed according to their own exercise formula.

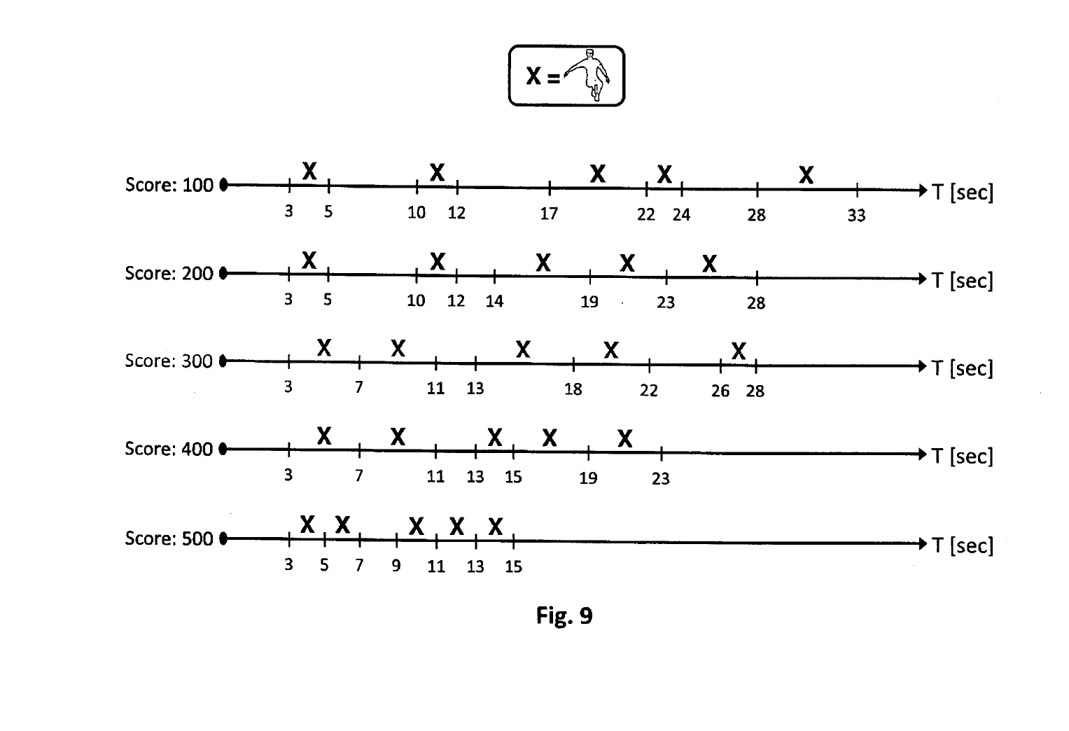

Reference is now made to FIG. 9, which shows an example of an exercise formula for a double leg jump, on a schematic timeline. Each sub-section has a score, and the double leg jump instances are depicted on the timeline by X marks. For example, in the first sub-section, the patient performs 3 seconds idle time, one double leg jump within the next 2 seconds, 5 seconds idle, one double leg jump within the next 2 seconds, etc. After finishing the first sub-section (and first 5 double leg jumps), if the patient has more than 5 double leg jumps prescribed in his therapy plan, he then may perform the second sub-section, etc, As one can see, the action intensity may increase upon advancing in sub-sections. Moreover, activity and idle sections may alternate within the sub-sections.

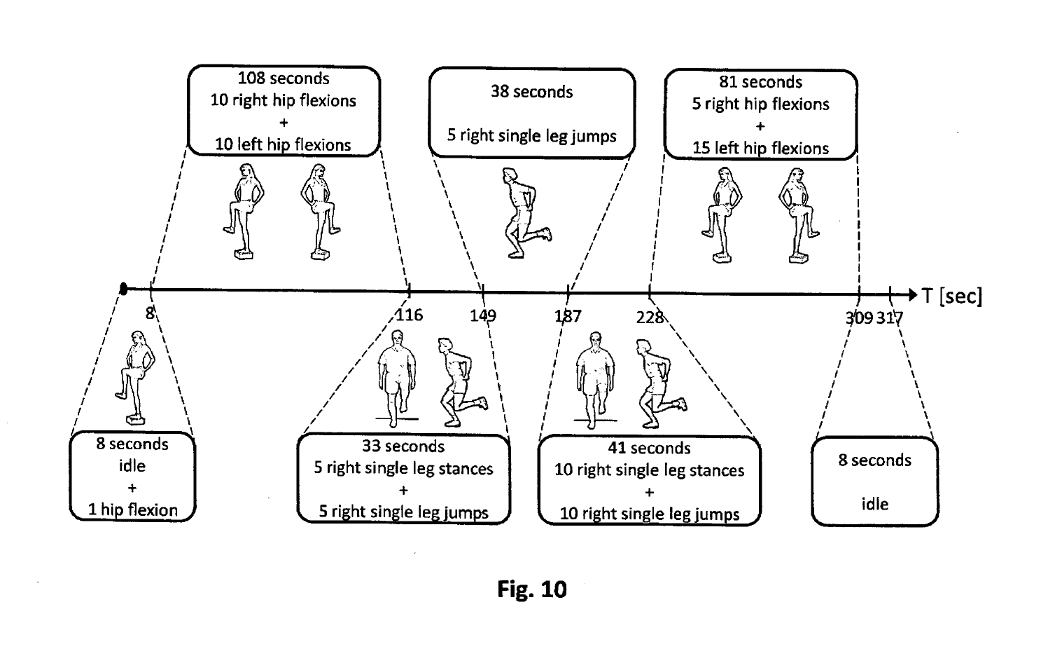

Reference is now made to FIG. 10, which shows an example of a complete, automatically-generated video game level, on a schematic timeline, in accordance with an exemplary scenario. The level may be built according to the level formula and exercise formulas described hereinbefore. In this specific example, the therapist prescribed the following plan to the patient: right hip flexion—5 repetitions, left hip flexion—5 repetitions, right single leg stance—5 repetitions, and right single leg jump—20 repetitions, all of the exercises are to be performed with medium intensity.

According to the level formula (described in connection with FIG. 2), the level may start with 8 seconds of idle time, except for one hip flexion, to imply the dominant exercise in this level.

The following section may include 30% of the prescribed hip flexions, i.e. 10 right hip flexions and 10 left hip flexions, performed for both left and right sides alternately, to avoid a long repeated and boring one-side workout. The exercise formula for hip flexion for two sides (described hereinbefore in FIG. 4) may then take action. The exercise may start at score 0 since it is medium intensity. After finishing the 100 sub-section (including 5 right and 5 left hip flexions), the score may be 100 and thus the next sub-section, namely—the 200 sub-section, is appended, to complete the remaining 5 right and 5 left hip flexions. The section may last 108 seconds (63 seconds for 100 sub-section+5 seconds for 200 sub-section).

The following section may include 40% of the prescribed single leg stances, i.e. 5 single right leg stances. Since there are also single leg jumps prescribed, each single leg stance may be immediately followed by a single leg jump. The exercise formula for single leg stance for one side (described hereinbefore in FIG. 5) may then take action. The exercise may start at score 0 since it is medium intensity. The patient may perform only the 100 sub-section to complete the 5 right leg stances. The section may last 33 seconds (33 seconds for 100 sub-section).

The following section may include 20% of the prescribed single leg jumps, i.e. 5 single right leg jumps. The exercise formula for a single leg jump for one side (described hereinbefore in FIG. 7) may then take action. The exercise may start at score 0 since it is medium intensity. The patient may perform only the 100 sub-section to complete the 5 right leg jumps. The section may last 38 seconds (38 seconds for 100 sub-section).

The following section may include the remaining 60% of the prescribed single leg stances, i.e. 10 single right leg stances. Since there are also single leg jumps remaining, each single leg stance may be immediately followed by a single leg jump. The exercise formula for single leg stance for one side (described hereinbefore in FIG. 5) may then take action. The exercise may start at score 100 since the 100 sub-section was already completed in a previous section. After finishing the 200 sub-section (including 5 right single leg stances+5 right single leg jumps), the score may be 300 and the patient may perform the 300 sub-section to complete the remaining 5 right single leg stances and 5 right single leg jumps. The section may last 41 seconds (23 seconds for 200 sub-section+18 seconds for 300 sub-section).

The following section may include the remaining 70% of the prescribed hip flexions, i.e. 5 right hip flexions and 5 left hip flexions, performed for both left and right sides alternately, to avoid a long repeated and boring one-side workout. The exercise formula for hip flexion for two sides (described hereinbefore in FIG. 4) may then take action. The exercise may start at score 200 since the 200 sub-section was already completed in a previous section. After finishing the 300 sub-section (including 5 right and 5 left hip flexions), the score may be 400. Since there are only left hip flexions remaining, the exercise formula for hip flexion for one side (described hereinbefore in FIG. 3) may then take action and the patient may perform the 400 sub-section to perform the remaining 10 left hip flexions. The section may last 81 seconds (63 seconds for two side 300 sub-section+18 seconds for one side 400 sub-section).

The level may end with 8 seconds of idle time.

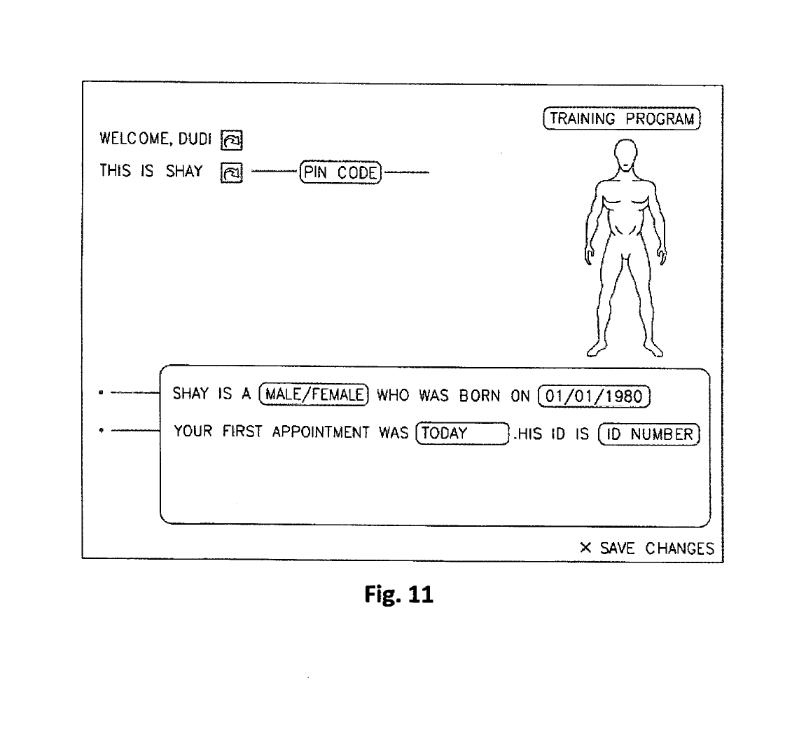

Reference is now made to FIG. 11, which shows an example of a dedicated web site page which summarizes information on a certain patient for the therapist. The page may display a summary of the patient profile, appointments history, diagnosis, other therapists comment history, etc.

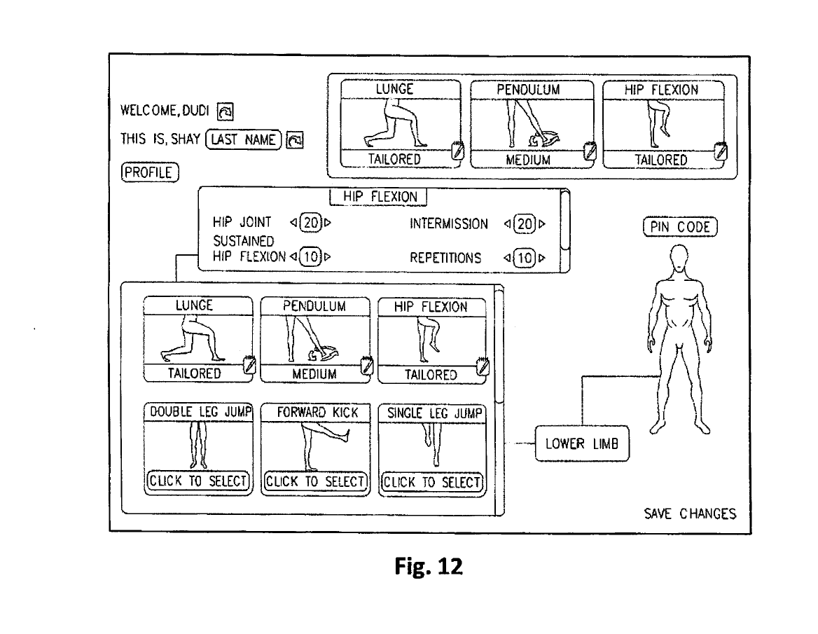

Reference is now made to FIG. 12, which shows an example of a dedicated web site page which is utilized by the therapist to construct a therapy plan for a certain patient. The therapist may input the required exercises, repetition number, difficulty level, etc.

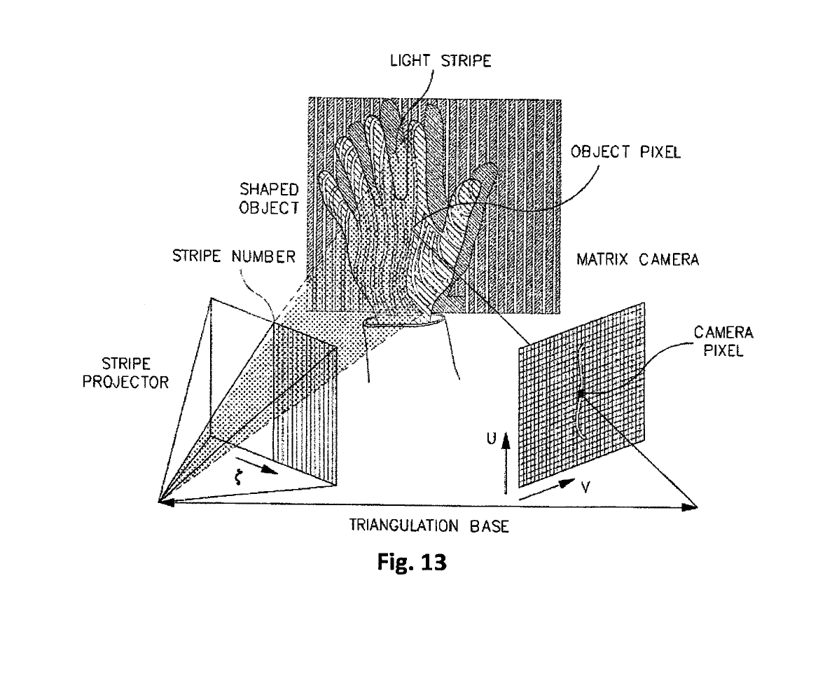

Since the use of motion recognition device may be significant for the present method, the principle of operation of a commercially-available motion recognition device (Kinect) and its contribution to the method is described hereinafter. Reference is now made to FIG. 13, which shows an illustration of a structured light method for depth recognition. A projector may be used for projecting the scene with known stripe-like light pattern. The projected object may distort the light pattern with equivalency to its shape. A camera, which may be installed at a known distance from the projector, may then capture the light reflected from the object and sense the distortion that may be formed in the light pattern, and the angle of the reflected light, for each pixel of the image.

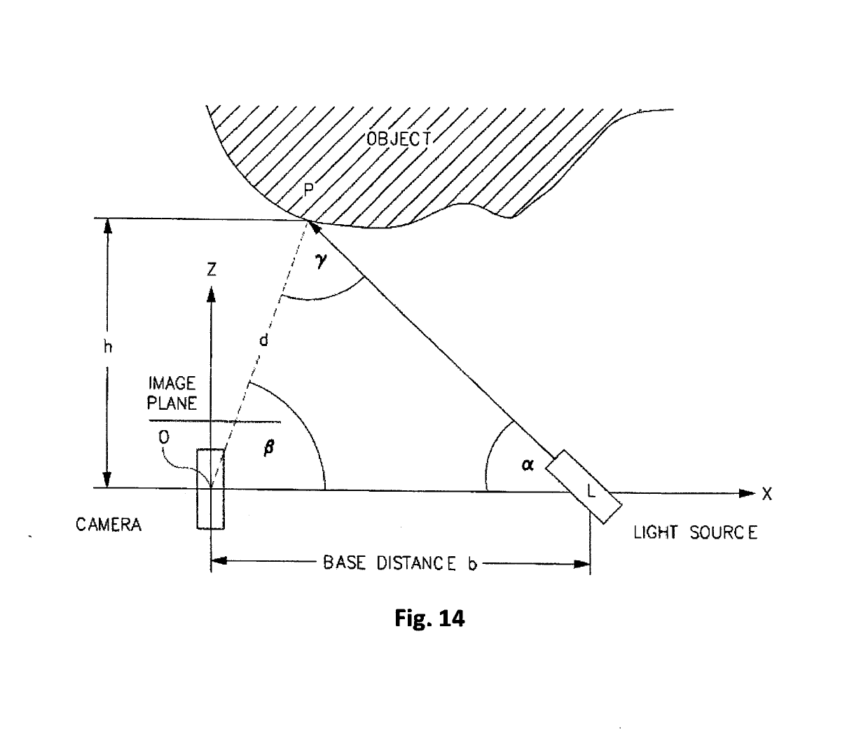

Reference is now made to FIG. 14, which shows a top view 2D illustration of a triangulation calculation used for determining a pixel depth. The camera may be located in a known distance from the light source (b). P is a point on the projected object which coordinates are to be calculated. According to the law of sines:

$$\frac{d}{sin α} = \frac{b}{sin γ} {}^{yields} d = \frac{b \cdot sin α}{sin γ} = \frac{b \cdot sin α}{sin ( π - α - β )} = \frac{b \cdot sin α}{sin ( α + β )}$$

and P coordinates are given by (d cos β, d sin β). Since α and b are known, and β is defined by the projective geometry, P coordinates may be resolved. The above calculation is made for 2D for the sake of simplicity, but the real device may actually calculate a 3D solution for each pixel coordinates to form a complete depth image of the scene, which may be utilized to recognize human movements.

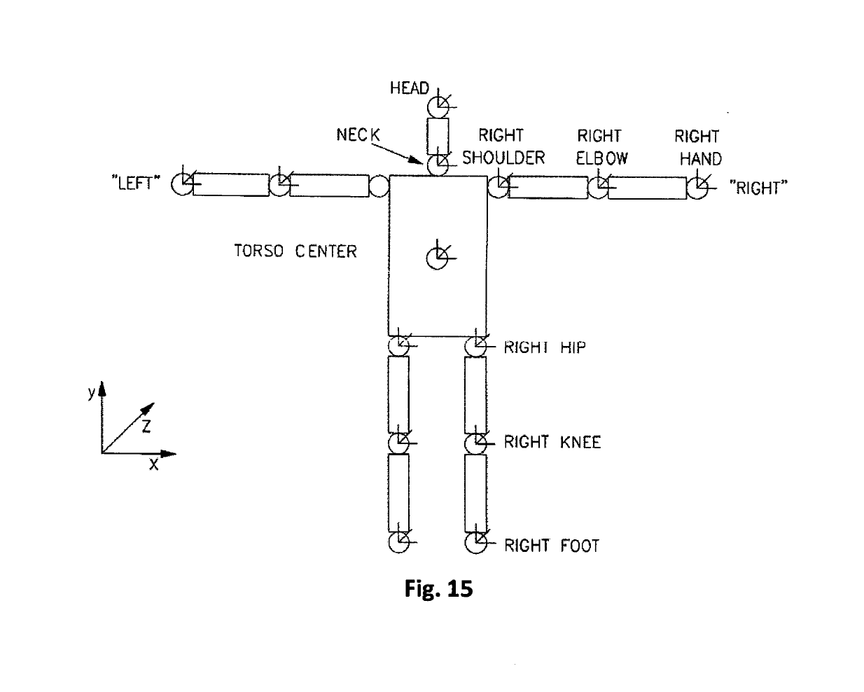

Reference is now made to FIG. 15, which shows an illustration of human primary body parts and joints. By recognizing the patient body parts and joints movements, the discussed method may enable to analyze the patient gestures and responses to the actions required by the game, for yielding an immediate feedback for the patient, and for storage for future analysis by the therapist.

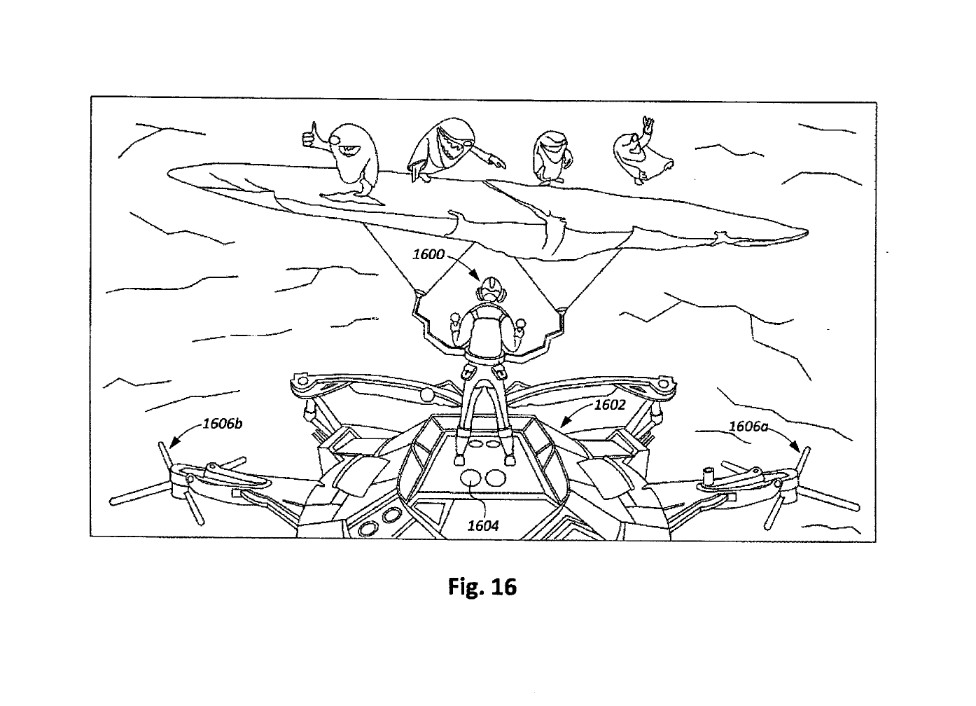

Reference is now made to FIG. 16, which shows one example of a video game level screen shot. This specific level may be designed to include squats, lunges, kicks, leg pendulums, etc. The patient may see a character (1600) performing his own movements at real time. Character (1600) may stand on a moving vehicle (1602), which may accelerate when the patient is performing squats, and may slow when the patient lunges. Some foot spots (1604) may be depicted on vehicle (1602) platform and may be dynamically highlighted, in order to guide the patient to place his feet in the correct positions while performing the squats, lunges, kicks, etc. Right and left rotating devices (1606a and 1606b, respectively) may be depicted on the right and left sides of vehicle (1602), to form a visual feedback for the patient while performing leg pendulum exercises.

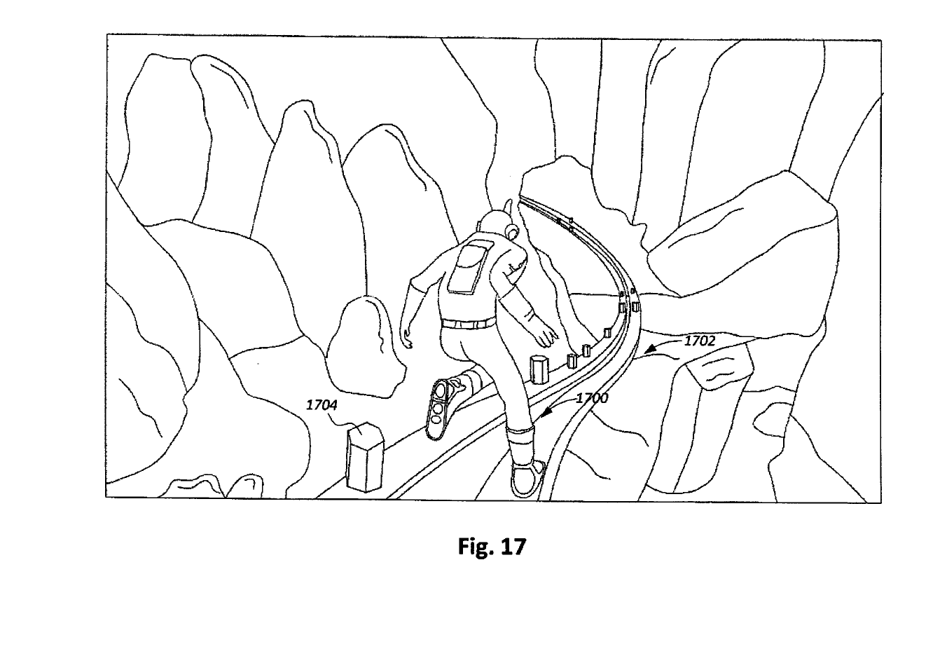

Reference is now made to FIG. 17, which shows another example of a video game level screen shot. This specific level may be designed to include hip flexions, leg stances and jumps, etc. The patient may see a character (1700) performing his own movements at real time. Character (1700) may advance on a rail (1702) planted with obstacles (1704). The patient may need to perform actions such as hip flexion, leg jump, etc., to avoid the obstacles and/or collect objects.

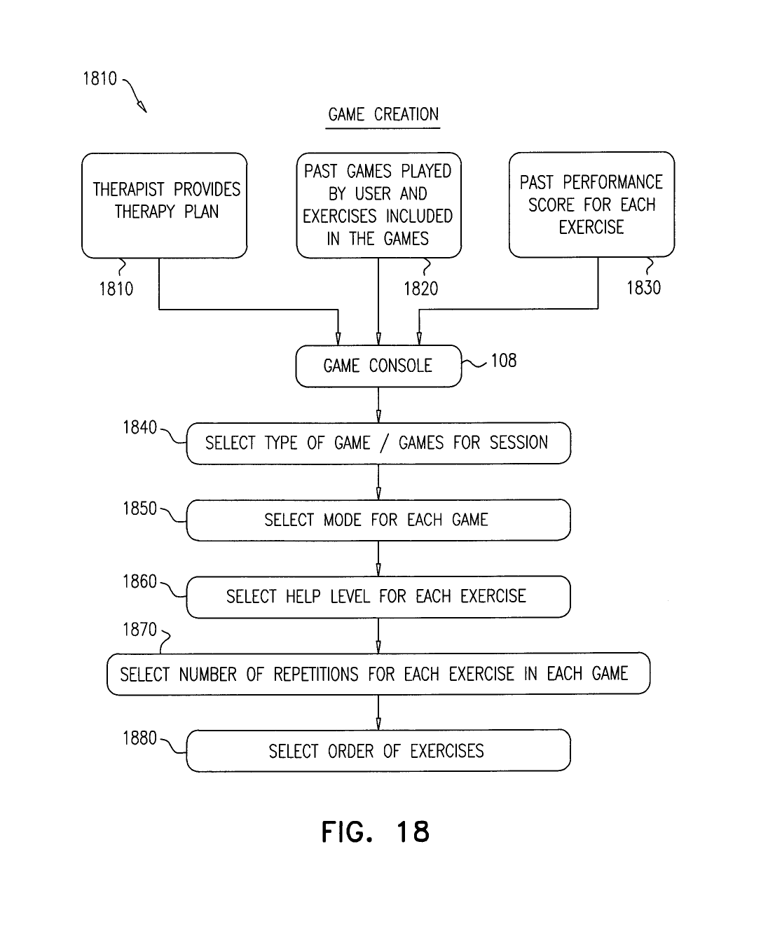

FIG. 18 shows a schematic flow diagram 1800 of a method of creating a user specific therapeutic game. In an exemplary embodiment of the disclosure, the therapist provides a therapy plan 1810, for example via web site 104 to gaming console 108. Optionally, the therapy plan includes a list of exercises and a number of repetitions for the user to execute each exercise as part of the therapy plan. In an exemplary embodiment of the disclosure, a list of past games 1820 performed by the user and optionally, the exercises included in each game are also provided to gaming console 108. In some embodiments of the disclosure, a past performance score 1830 of the user for each exercise or each gesture of each exercise is also provided to gaming console 108.

In an exemplary embodiment of the disclosure, the above information is used to select the type of game 1840 or games for performing an exercise session in which the user performs the exercises required by the therapy plan. Optionally, each game is designed to support specific exercises (each exercise including one or more gestures). The gaming console 108 is programmed to select a set of one or more games that cover all the exercises required by the therapy plan 1810, so that by playing an exercise session the user will perform all the designated exercises in the therapy plan 1810. Optionally, the selected games may include additional exercises that were not required in the therapy plan to enhance enjoyment from the games, for example a user that needs therapy for his or her feet may also perform hand exercises although they were not part of the therapy plan. In some embodiments of the disclosure, the therapy plan may specify if the user has specific limitations that need to be taken into consideration by gaming console 108 besides the exercises that need to be performed, for example a limit on the work load that the user can perform or if specific limbs are not functional and cannot be included in the game. In an exemplary embodiment of the disclosure, in each game the exercises may be grouped together or mixed with the other exercises that were not required in the therapy plan. Each game may be divided into multiple levels and the exercises may be distributed, so that a portion of the exercises is performed in each level.

In an exemplary embodiment of the disclosure, gaming console 108 also selects a mode 1850 for each selected game. For example a game that is new for the user will be played in a beginner mode in which the user is provided with a full explanation of the game and each exercise. Optionally, in the beginner mode, user monitoring focuses on compliance to the requirements of the game and less on the quality of performing each gesture correctly. Optionally, if the user has already played the specific game in the past, gaming console 108 provides less guidance and requires stricter compliance in performing the exercises.

In some embodiments of the disclosure there are a few mode levels, for example beginner, experienced and veteran. Alternatively, there may be more mode levels for example gaming console 108 may keep a count for each user, the count representing a mode level of familiarity with the games (e.g. how many times the user played the game).

Optionally, in each mode level the gaming console 108 may require different levels of performance, for example in performing a single leg jump:

1. For a beginner user failure to jump or loss of balance may be considered failure;

2. For an experienced user even landing on both legs instead of one leg may also be considered failure; and

3. For a veteran user even bending knees may be considered failure.

In an exemplary embodiment of the disclosure, gaming console 108 also selects a help level 1860 for each exercise. Optionally, help may be provided by providing a written message on the game screen, by providing a verbal message, by providing a visual message (e.g. a video) or a combination thereof. For example a user that was supposed to jump that did not jump may be shown a message (e.g. “Jump not detected”) or receive a verbal message telling the user to jump higher or be shown a video in a corner of the screen or on the entire screen demonstrating the correct performance. The help may be provided during the game or the game may be paused to assure that the user gets the message. Optionally, when creating the game session gaming console 108 may establish a help level for each exercise and/or each gesture of the exercise. In an exemplary embodiment of the disclosure, the help level for each exercise is established based on the past performance of the user for the specific exercise. Optionally, help is triggered when the user fails to perform a specific exercise correctly. In some embodiments of the disclosure, help may be provided after a single failure or only after repetitive failures, for example failing to perform a specific exercise 3 times in a row or 8 times out of 11. Optionally, the help level may be increased if the user's performance for a specific exercise does not improve.

In an exemplary embodiment of the disclosure, gaming console 108 also selects a number of repetitions 1870 for each exercise in each game. Some exercises may only be available in some types of games, whereas some exercises may be available in most games. Optionally, gaming console 108 instructs each game as to what portion of each exercise from the therapy plan will be performed in each game, for example if the therapy plan requires performing straight leg raising 30 times, one game may be activated to perform the exercise 10 times and a second game may be activated to perform the exercise 20 times.

In an exemplary embodiment of the disclosure, gaming console 108 also selects an order 1880 for performing the exercises in each game. In some embodiments of the disclosure, the therapy plan may indicate which exercises should be performed first, to warm up the user's muscles. Alternatively or additionally, the order may be selected based on the performance of the user, for example starting with exercises that were easy for the user and advancing to exercises that were harder for the user. Optionally, the order provides a sequential list defining which exercises are to be performed, the number of times each exercise is performed in a row, the time allocated for performing each exercise and duration of breaks between each exercise. In some embodiments of the disclosure, the exercises in the games or in each level of a game may be arranged randomly to make the game more challenging by surprising the user with an unexpected game.

In some embodiments of the disclosure, web server 104 provides the computation power for performing the above described selections and gaming console 108 executes the games. Alternatively, gaming console 108 may receive the information from web server 104, for example either directly or via database 106, and perform the selections using a processor and memory in the gaming console 108.

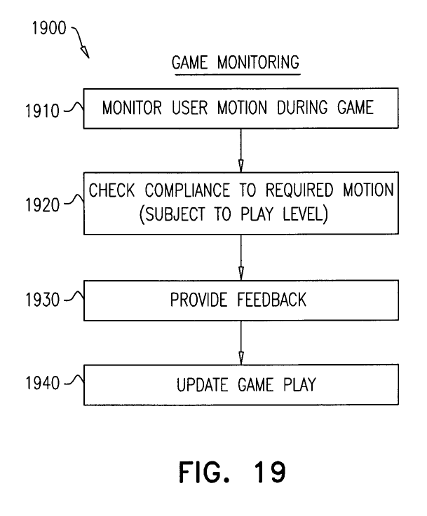

FIG. 19 shows a schematic flow diagram of a method 1900 of monitoring a user specific therapeutic game, in accordance with some embodiments. In an exemplary embodiment of the disclosure, during game play gaming console 108 monitors 1910 the user motion. Optionally, gaming console 1920 checks the user motion to verify compliance to the required motion. In an exemplary embodiment of the disclosure, gaming console 108 takes into account the game mode (e.g. beginner, experienced, veteran) and help level. Optionally a score is provided for each gesture of each exercise and a total score for each exercise.

In an exemplary embodiment of the disclosure, gaming console 108 provides feedback 1930 based on the results of checking the motion relative to the correct performance. Optionally, the feedback may be positive or negative, for example if the user reaches a high score by performing the gestures correctly and with the right timing, then the user will receive positive feedback. However if the user receives low scores then the user will receive negative feedback, for example explanations as to how to perform correctly or questions regarding the reasons for the problems the user is having to perform the exercises correctly. Optionally, the feedback may be in the form of a written message, a voice message, a visual pictorial message, a video or combination of the options. The feedback may notify the user of the problem, question the user for the reason and/or illustrate the correct way of performing the exercise.

In an exemplary embodiment of the disclosure, a lower level of feedback includes notifying the user that an exercise was not performed correctly, a higher level includes notifying the user what was wrong with performing the exercise and an even higher level includes teaching the user how to perform an exercise and even higher may be to query the user for the reason of failing to perform correctly. Optionally, the higher or lower level of feedback may be applied to specific exercises depending on the performance of the user.

In some embodiments of the disclosure, a lower level may be to provide feedback as a written message (e.g. a message flashing on the screen), a higher level may be to verbally notify the user (e.g. by a narrator), an even higher level may be to show a picture of what was performed wrong, even higher might be to show the user a video of correct performance of the exercise and even higher might be to query the user for the reason of failing to perform correctly and require the user to select a reason from a list of displayed options.

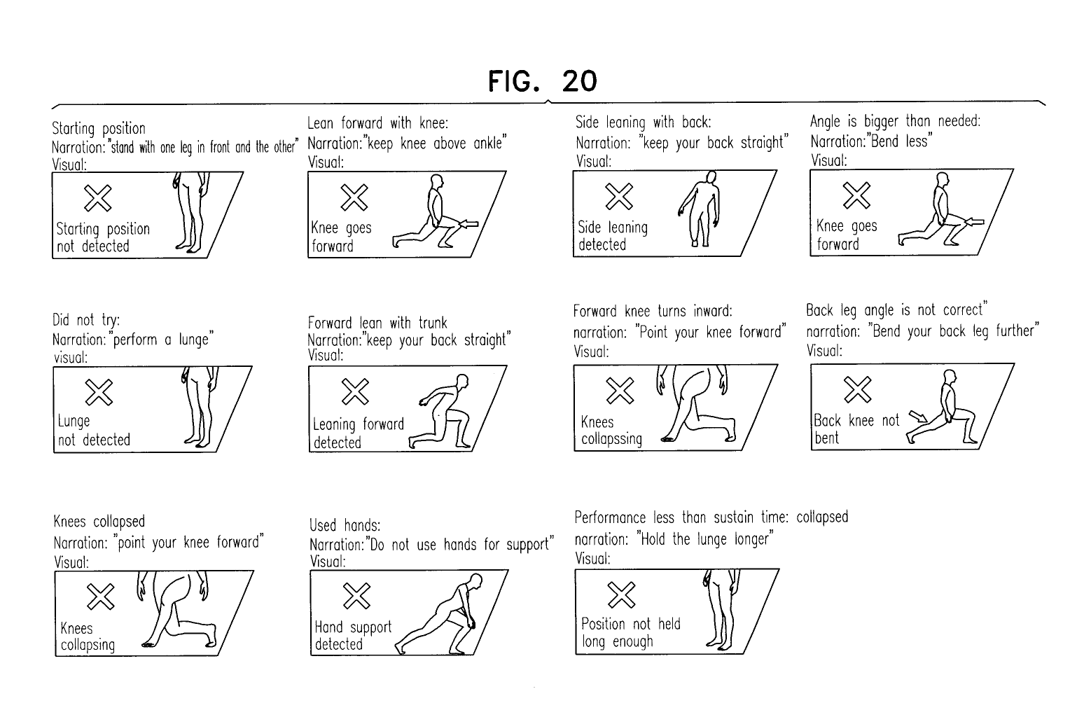

FIG. 20 shows a schematic diagram of feedback messages for a user when required to perform a specific exercise (e.g. a “Lunge”). Optionally, the feedback may be a message written on the screen, a verbal message spoken by a narrator, a visual message as in FIG. 20, a video showing the exercise/gesture or a combination of the possibilities.

As explained above the level of feedback might depend on the game mode, however during a game the level of feedback may be escalated responsive to the user's performance, for example if the user repeatedly makes the same mistake the level may be increased for the rest of the game or specifically for the problematic exercise. Likewise the level of feedback may be decreased if the user receives high scores repeatedly and the feedback seems unnecessary.

In an exemplary embodiment of the disclosure, the results of the monitoring are used to update the game-play 1940, for example to reduce or increase the number of times the user is required to perform a specific exercise, for example if the user does not manage to perform the specific exercise correctly. In some embodiments of the disclosure, a specific exercise may be canceled from the game and notification may be sent to the therapist. Optionally, the required timing for performing a specific exercise may be increased or decreased to fit the user's needs. The user's score may be updated accordingly.

In some embodiments of the disclosure, gaming console 108 may change the game plan in real-time, for example by canceling exercises, replacing harder exercises with easier ones, lowering the game mode or increasing the help level. Optionally, the entire game may be halted, for example if the user falls over or stops responding.

In an exemplary embodiment of the disclosure, at the end of a game or if the user completely fails to perform specific exercises, gaming console 108 may present the user with a question as to why he failed to perform. The user answers may include:

1. That the exercise is too difficult.

2. That the user does not understand the game.

3. That the user does not understand the exercise.

Optionally, the user's answers may be provided to the therapist to revise the therapy plan or the answers may be provided directly to the gaming console 108 to be considered when preparing a new game for the user, for example when considering the list of past games 1820 or past performance for exercises. In an exemplary embodiment of the disclosure, a new game may include more or less repetitions of specific exercises. In some embodiments of the disclosure, in the new game the user may receive special care (at least for one game), for example by providing the maximum help level and/or monitoring the user's performance in the lowest operation mode (e.g. beginner). In some embodiments of the disclosure, the user will be required to perform a tutorial before being able to start a new game. Optionally, the tutorial may be instead or in addition to playing with special care.

In an exemplary embodiment of the disclosure, if the user falls or performs very poorly relative to previous games then gaming console 108 may halt the game until the user's therapist forms contact with the user and reopens the gaming console 108 for use by the user. Optionally reopening the gaming console 108 is performed on web site 104 by the therapist and a notification is transmitted to gaming console 108.

In an exemplary embodiment of the disclosure, an exercise/gesture tutorial may include:

1. A narrator announcing the name of the exercise/gesture.

2. Explaining how to perform the exercise/gesture and demonstrating the performance on the screen.

3. Testing the user in performing the exercise/gesture a few times, for example 3-10 times while monitoring the user to determine if the user performs the exercise/gesture correctly and providing feedback to the user.

Optionally, a game tutorial may include:

1. A narrator announcing the name of the game.

2. Explaining the objectives of the game (e.g. how to accumulate points and how to win) and how to perform the game optionally with a demonstration on the screen.

3. Testing the user's ability to perform some of the exercises/gestures that occur in the game, for example 3-10 times while monitoring the user to determine if the user performs the exercise/gesture correctly.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

What is claimed is:

1. A method for user specific, rehabilitative video gaming, the method comprising:
receiving the following information:
a. a rehabilitative therapy plan for the user, the rehabilitative therapy plan comprising multiple different physical exercises;
b. a list of past games played by the user and exercises included in the games to determine a level of familiarity of the user with the games and exercises;
c. past performance scores for each exercise performed by the user;
automatically translating the received information into a set of video games configured for execution by a motion recognition gaming system, wherein said automatically translating comprises:
selecting a set of one or more games that include all the exercises required by the therapy plan;
selecting a mode of operation for execution of the games by the user, based on the level of familiarity of the user with the games;
selecting a help level for each exercise in the game, based on the past performance scores; and
enabling the user to conduct an interactive gaming session using the selected games, by operating the motion recognition gaming system to recognize motion of the patient.

2. The method according to claim 1, wherein the set of one or more games include exercises that were not required by the therapy plan.

3. The method according to claim 1, wherein the translating takes into account user limitations to exclude certain types of exercises from the set of one or more games.

4. The method according to claim 1, wherein the modes of operation include a beginner mode, an experienced user mode and a veteran mode.

5. The method according to claim 1, wherein the mode defines a level of providing the user with guidance during use of the games.

6. The method according to claim 1, wherein the mode defines a level of requiring compliance with the requirements of the game and a level of required accuracy in performing the exercises.

7. The method according to claim 1, wherein an exercise in which the user has a high score will have a low help level and an exercise in which the user has a low score will have a high help level.

8. The method according to claim 1, wherein the help level of an exercise determines if the help for the exercise will be provided as a written message on a screen, a verbal message, a visual pictorial message, a video or a combination of the above.

9. The method according to claim 1, wherein said translating determines a portion of repetitions for performing each exercise from the therapy plan for each game from the set of one or more games.

10. The method according to claim 1, wherein said translating determines an order for performing exercises for each game.

11. The method according to claim 10, wherein the order of exercises in each game is set randomly to provide an unexpected order for the user.

12. The method according to claim 1, wherein the one or more physical exercises are selected from the group consisting of: hip flexion, jump, single-leg jump, leg stance, squat, single-leg squat, lunge, side lunge, forward kick, leg pendulum, lateral weight transfer, forward punch, shoulder flexion, shoulder abduction, shoulder upward press, side arm circle, diagonal arm movement, side punch, shoulder rotation with hands on body sides, shoulder rotation starting with abduction, and elbow flexion.

13. The method according to claim 1, wherein said receiving and said automatically translating are performed by a web server accessible by (a) a therapist who prescribes the rehabilitative therapy plan and (b) the motion recognition gaming system.

14. A non-transitory computer readable medium for storing a computer program for executing with a processor and memory the method of claim 1.

15. A computing system configured for user specific, rehabilitative video gaming, the system comprising:
a communication module configured to receive:
a. a rehabilitative therapy plan for the user, the rehabilitative therapy plan comprising multiple different physical exercises;
b. a list of past games played by the user and exercises included in the games to determine a level of familiarity of the user with the games and exercises;
c. past performance scores for each exercise performed by the user;
a non-transient memory comprising instructions for automatically translating the therapy plan to a video game configured for execution by a motion recognition gaming system, wherein said automatically translating comprises:
selecting a set of one or more games that include all the exercises required by the therapy plan;
selecting a mode of operation for execution of the games by the user, based on the level of familiarity of the user with the games;
selecting a help level for each exercise in the game, based on the past performance scores; and
a hardware processor configured to execute the instructions, so as to generate the video games.

16. The computing system according to claim 15, wherein said communication module is further configured to transmit the video games to the motion recognition gaming system.

17. The computing system according to claim 15, wherein the computing system further comprises a motion recognition gaming system to recognize motion of the patient and conduct an interactive gaming session with the user using the selected games.

18. The computing system according to claim 15, wherein said translating determines a portion of repetitions for performing each exercise from the therapy plan for each game from the set of one or more games.

19. The computing system according to claim 15, wherein said translating determines an order for performing exercises for each game.

20. The method according to claim 19, wherein the order of exercises in each game is set randomly to provide an unexpected order for the user.
